(19) United States
(12) Patent Application Publication
(10) Pub. No.: US 2016/0283696 A1
(43) Pub. Date: Sep. 29, 2016

(54) Methods and device arrangement for physical activity thresholds promoting fat and cholesterol metabolism in high risk subjects

(71) Applicant: Juhani LEPPÄLUOTO, Oulu (FI)

(72) Inventors: Juhani Leppäluoto, Oulu (FI); Karl-Heinz Herzig, Oulu (FI); Riikka Ahola, Oulu (FI); Timo Jämsä, Oulu (FI); Jari Jokelainen, Oulu (FI); Sirkka Keinänen-Kiukaanniemi, Oulu (FI)

(21) Appl. No.: 15/034,185

(22) PCT Filed: Oct. 31, 2014
(86) PCT No.: PCT/FI2014/000031

§ 371 Date: May 4, 2016

(30) Foreign Application Priority Data
Nov. 4, 2013 (FI) 20130323

(51) Int. Cl. G06F 19/00; A61B 5/11

(52) U.S. Cl. CPC G06F 19/3481; A61B 5/1118; A61B 2562/0219

(57) ABSTRACT

Method and device determine the threshold values for the volume and intensity of physical activity or the lowest levels that reduce risk factors in persons who suffer from disorders of sugar and lipid metabolism and are overweight. The method characterized in that the device registers a person's daily physical activity, compares it with the measured threshold levels and informs by its own or outside screen, if the volume and/or intensity of the physical activity has been sufficient to prevent the effects of disease risk factors. Based on this persons, who have disorders in sugar and lipid metabolism are able to perform physical activity maintaining health. The invention described herein demonstrates as a novel observation that even the low intensity physical activity will restrain risk factors related to cardiovascular diseases, diabetes and obesity.

The invention relates to method and device for determining physical activity thresholds to improving sugar, cholesterol and fat metabolism in subjects who have sedentary life style. The use of motion sensors and transduction units to measure physical activity intensity and volume have been presented in U.S.2008/0312560A1, WO02005117703. A method in which physical activity improving cholesterol metabolism in healthy subjects is presented in U.S. 2010/0137107 A1.

The invention described here belongs to the medical discipline and includes the determination of physical activity thresholds that can be used for preventing risks for metabolic diseases such as diabetes and cardiovascular disease by reducing blood cholesterol, triglycerides and visceral fat mass. The earlier invention purposed for healthy subjects related to physical activity reducing blood cholesterol (U.S. 2010/0137107 A1) is not suitable to subjects who have risks of diabetes and/or cardiovascular diseases, as they are not capable to perform prescribed physical activity previously shown to reduce blood cholesterol in healthy subjects (Herzig et al. 2014 Int J Obesity38:1089).

### THE BACKGROUND OF THE INVENTION

Overweight and metabolic diseases increase significantly in industrialized countries in which energy rich diets are widely used and energy expenditure remains low. According to the WHO statistics over 1,400,000,000 of the world population are overweight or obese. Overweight and low physical activity result in an increase in the prevalence of metabolic diseases mainly diabetes and cardiovascular diseases. It is currently estimated that 500,000,000 of the population suffer from type 2 diabetes and the number will be doubled by the year 2030. In addition atherosclerosis, calcification of blood vessels, due to sedentary life style exposes to deaths for cardiovascular diseases being the most frequent death causes in industrialized countries. The dead roll is presently 15,000,000 annually and is growing every year. Low levels of physical activity is also a dangerous risk factor disability-adjusted life years. According to the present WHO statistics it is annually responsible for 1,000,000 death cases and loss of 8,000,000 disability-adjusted life years.

Official Guidelines for Physical Activity Promoting Health

The present physical activity recommendations of WHO, American Diabetes Association and American Heart Association state that healthy adults should perform at least 150 min moderate intensity physical activity every week such as walking at the speed of 5 km/h. This threshold should imply walking 2 km or 3000 steps within 20 min during all days of the week. These recommendations have been developed by reviewing subjects about their physical activity levels and demonstrated that about 50% of the adult population met the guidelines. Later physical activity levels have been studied objectively by accelerometers and the results showed that only less than 5% of the adult population will meet the above-mentioned official guidelines of physical activity. Therefore all the previous studies about the health effects of physical activity using personal reviews or questionnaires are questionable.

Physical Activity and Risks for Diabetes, Cardiovascular and Metabolic Diseases

Calcification of blood vessels, high blood sugar, cholesterol and triglycerides are risk factors for diabetes and cardiovascular diseases. These risk factors can be reduced by increased physical activity and weight reduction programs. Increased exercise will lead to decreases in blood LDL-cholesterol and increases HDL-cholesterol (tannescu et a. 2002 JAMA, 288,4994, Kraus et al. 2002, NEJM 347, 1483) all reducing risks of cardiovascular diseases. In a similar way increased weekly exercise and weight reduction programs prevented developing of the type 2 diabetes in 60% of patients (Tuomilehto et al. 2002,NEJM 304.1343, Knowler et al. 2002, NEJM 346:393). Fat tissues especially those around the internal organs are body's energy sources and are released to blood circulation during exercises. It has been shown that during exercises fat from the internal organs is first mobilized and starts the reduction of body weight (O'Leary et al. 2009, J Appl Physiol 100,1958).

The results of scientific studies demonstrate that increased physical activity is effective in preventing risk factors of diabetes and cardiovascular diseases and is an important vehicle in weight reduction. However, in none of these cited or other previous studies the exact amounts and intensities of physical activity preventing diabetes or cardiovascular diseases have been described. Our research groups is the only one that have measured physical activities by objective methods in order to find out beneficial effects of disease risk factors. Our main goal has been to define the minimum amounts and intensities or threshold levels which will reduce the risks for diabetes and cardiovascular diseases. By using our accelerometer we have previously demonstrated that blood cholesterol decreases by 15% in healthy women as the number of daily steps exceeded 1000 at acceleration of 2.2 g corresponding to walking at the speed of 5 km/h (Vainionpää et al. 2007, Me Sci Sport Exerc 39,756).

Scientific Research Related to the Invention

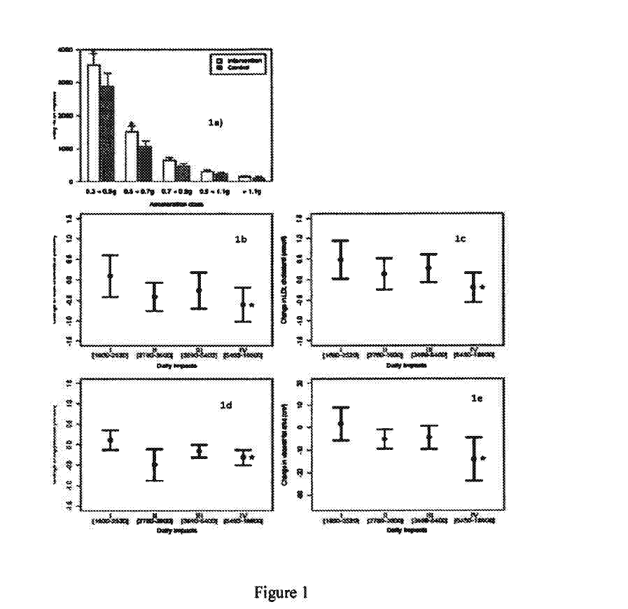

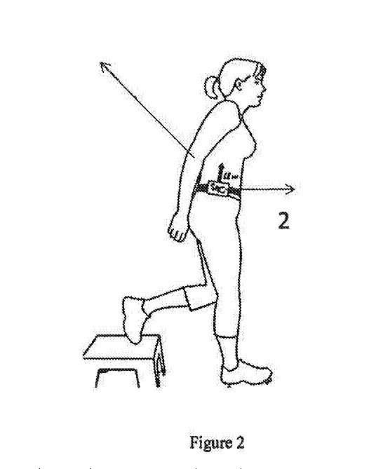

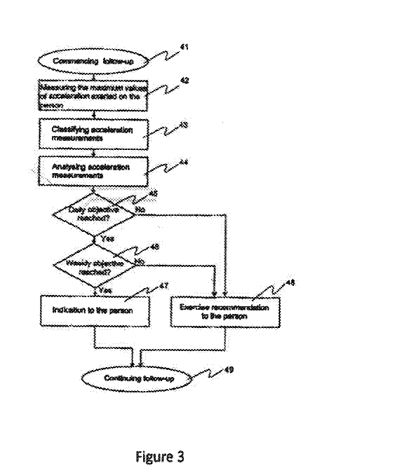

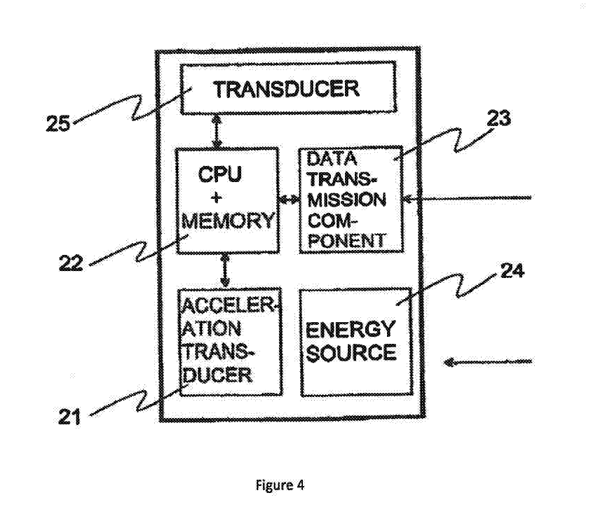

In our study related directly to this intervention we observed that in overweight subjects having abnormal sugar balance clearly lower intensity levels of physical activity than those given in the official physical activity recommendations reduced risks for diabetes and cardiovascular diseases (Herzig et al.2014, Int J Obesity 38:1089). Walking at accelerations of 0.3-0.7 g corresponding to the walking speed of 2-3 km/h led to decreases in blood triglycerides and cholesterols (FIG. 1A) and visceral fat (FIG. 1B) as the number of daily steps exceeded 6520. This was a very important finding, since most overweight subjects suffering from abnormal blood glucose are not able to walk at the speed of 5 km/h required in the present official recommendations. Therefore most of these subjects do not follow the present recommendations. Our accelerometer recording amounts and intensities made it possible to determine exact threshold levels of physical activity promoting health to be used in the reduction of risk factors of metabolic diseases.

To Which Problem Does Our Invention Offer the Best Solution?

High occurrence of overweight, obesity, diabetes and cardiovascular diseases in the populations of industrialized countries are major health problems. Precise physical activity guidelines are not available in adequately targeting these health problems. Our present intervention offers exact information about amounts and intensities of physical activity resulting in reduction of risks for diabetes and cardiovascular diseases and visceral fat.

Previous Solutions

Before our present invention there was no information about the exact amount and intensity of physical activity reducing risk factors of diabetes and cardiovascular diseases in subjects suffering from metabolic disorders. The present physical activity guidelines (WHO, American Diabetes Association and American Heart Association) are based on questionnaires and reviews from healthy subjects and are too demanding for most general subjects often having overweight and suffering from metabolic disorders. Blood sugar and cholesterol levels have been previously followed by motion sensors in subjects who have abnormal blood glucose but the sensors have been used only for the following of the physical activity but not for the studies of health effects (Yates et al. 2009, Diabetes care 32:1404, Saito et al. Ann Intern Med 2011, 171,1352). In healthy subjects the amounts and intensities of physical activity reducing blood cholesterol (Vainionpää et al. 2007, Med Sci Sports Exerc.39,756, U.S. patent 2008/0312560 A1) have been solved by accelerometers detecting number of steps and intensity (g-values). In the same subjects the amount and intensity of the physical activity preventing osteoporosis was solved (Vainionpää et a. 2006, Int J Osteoporosis 17,455, US patent, US 2010137107 A1). It should be noted that both studies deal on the same subjects, to whom physical activity thresholds reducing cholesterol and preventing osteoporosis could be defined simultaneously by the accelerometer.

The invention described here manages physical activity thresholds reducing blood cholesterol, triglycerides and visceral fat in overweight subjects with metabolic disorders. These subjects are not able to reach physical activity thresholds presented in the patent (U.S. 20107137107), and therefore they need their own physical activity thresholds. The patents U.S. 2008/0312560 A1 and U.S. 2010/137107 A1 and the one described here belong to the same patents family, in which determining of physical activity thresholds are novel and differ from the previously known techniques

Accelerometers (patents U.S. 2010191155 A1, EP 0700661 A2, U.S. 2009171614 A1, EP 2210557 A1 and U.S. 2001049470 A1) and a heart rate monitor (U.S. 20110213) have been previously used for measuring energy expenditure of physical activity or following body weight and nutrient intake in healthy subjects and heart rate monitor for protein consumption and recovery in athletes. It should be noted that all the above mentioned methods measuring energy expenditure are useless for establishing threshold levels improving health. For instance in a previous study we demonstrated that walking 12982 daily steps at the acceleration range 0.3-1.0 g had no effect on blood cholesterol, whereas 1062 daily steps at the acceleration range 1.1-2.4 g reduced blood cholesterol by 15% (Vainionpää et al. 2007, Med Sci Sports Exerc 39,756). 12982 daily steps at 0.3-1.0 g stand for 10 MET-units, but 1062 daily steps at 1.1-2.4. stand for 1 MET-unit. We therefore conclude that energy expenditure in MET-units or calories which have been used in the above mentioned patents, neither relate to determining thresholds nor health benefits of physical activities in sedentary subjects.

Our invention has many advantages. It makes it possible to determine for the first time the volume and intensity of the physical activity threshold reducing disease risks in subjects, who are sedentary, have overweight and suffer from metabolic disorders. These subjects cover approximately 50% of the population and previously there have not been any physical activity recommendations for them. The accelerometer described in the intervention can be easily used continuously making it possible to register all types of physical activity including habitual activities. This will make it easy to meet our novel physical activity recommendations. From the point of preventive medicine walking is also the best form of physical activity. It activates the biggest muscles in the body and its energy expenditure can be accurately measured by accelerometers recording number of steps and step induced accelerations or walking speeds. Maintaining walking ability protects also from falling accidents and hospitalization.

High occurrence of overweight, obesity and metabolic disorders in the present populations and the information of the preventive characteristics and efficacy of the invention described here will offer large possibilities to its extensive use and to commercial adaptations.

### THE METHOD OF THE INVENTION

The results presented in FIG. 1A and 1B are derived from 68 subjects carrying accelerometer for 3 months. The subjects were overweight men and women aged 30-70 years and had abnormal blood sugar. One half of the subjects participated in supervised exercise weekly and the other half continued their normal life style. The subjects carried accelerometers during wake-full time on their waist. The acceleration values their daily numbers were continuously registered. Blood samples were taken at the beginning and end of the trial to measure blood sugar, insulin, cholesterol, LDL-cholesterol and triglycerides. The amount of the visceral fat was measured by a bio-impedance method. The distribution of the accelerations during 3 months showed that exercises increased significantly number of steps or impacts in the acceleration classes 0.3-0.7 g, corresponding typically to slow walking 2-3 km/h. The steps in the acceleration class 0.3-0.7 were divided by their magnitudes to quartiles. Cholesterol and fat concentrations between each quartile were analyzed. The results showed (FIG. 1A and 1B) that total cholesterol, LDL-cholesterol, triglycerides, and visceral fat in the fourth quartile (over 6520 daily steps) were significantly lower than in the other quartiles. 6520 daily steps is the threshold for physical activity, the exceeding of which reduced total cholesterol by 0.6 mmol/l, LDL-cholesterol 0.7 mmol/l, triglycerides 0.4 mmol/l and the area of visceral fat by 12%. Our results show that physical activity volume and intensity measured by the invention described herein explain beneficial health effects observed in cholesterol and triglyceride concentrations and in visceral fat. Corresponding results in subjects with sedentary life style have not been able to obtain by using previous methods.

The invention described herein does essentially differ from the previously known methods by which exercises have been used for prevention of risk factors of diabetes and cardiovascular diseases. Physicians have generally been aware about the beneficial effects of regular exercise, but the volume, intensity and duration of the exercises have been unknown. It was novel and unexpected in our invention that in sedentary subjects physical activity of very low intensity (accelerations 0.3-0.7 g) reduced concentrations of the major risk factors of diabetes and cardiovascular diseases. Our invention is also different form other patents and recommendations, since those do not take low intensity exercise into account.

1. A method for defining and presenting the threshold levels of physical activity for reducing blood cholesterols, triglycerides and visceral fat, the method comprises:
a step, in which by a transducer unit (2) carried by the person (1) accelerations of a body of the person (1) induced by physical activity are registered;
a step (43) in which occurrence numbers (N) of registered acceleration maxima within a certain time period are classified and stored according to the maxima magnitude to one of four acceleration classes;
a step (44) in which the numbers of acceleration maxima of the person (1) in each acceleration class are compared to the reference data measured from exercising persons' numbers of acceleration maxima in each acceleration classes; and
a step (45, 46) in which by using the number of acceleration maxima in the acceleration classes it is identified into which physical activity class the person can be classified,
characterized in that the method further comprises:
a step (47, 48) in which it is identified and presented (1) that an amount of the physical activity performed is classified to a physical activity classes that are:
beneficial for sugar metabolism, cholesterol metabolism and decrease of visceral fat; or
a recommendation of additional physical activity for a sedentary an overweight person.

2. The method according to claim 1, characterized in that when occurrence numbers (N) of physical activity of the person (1) exceeds a threshold value of an acceleration maxima of a particular acceleration class it is presented that the exercise performed has reduced disorders in sugar, cholesterol and fat metabolism.

3. The method according to claim 2, characterized in that for defining the highest exercise activity class the registered acceleration maxima are divided into several acceleration classes between 0.3 and 10 g (0 g corresponds standing), into classes 0.3-0.5 g; 0.5-0.7 g; 0.7-0.9 g; 0.9-1.1 g and >1.1 g.

4. The method according to claim 3, characterized in that each measured acceleration maxima adds to the occurrence number (N) by one in the acceleration class to which it belongs.

5. The method according to claim 4, characterized in that when the daily occurrence number (N) of acceleration maxima exceeds the given threshold of 6520 daily steps within the acceleration class 0.3-0.7 g, the person (1) has performed physical activity, which has reduced blood cholesterols, triglycerides and visceral fat.

6. A transducer unit (25) comprising:
a uni- or polyaxial accelerometer (21) that continuously register accelerations maxima induced by a physical activity of a person (1);
a memory for storing the acceleration maxima; and
a processing unit (22) configured to classify the acceleration maxima into acceleration classes between 0.3 g and 10 g,
characterized in that it is further configured to:
compare the classified acceleration maxima counts of the person (1) with a threshold of 6520 daily steps in the acceleration classes 0.3-0.5 g and 0.5-0.7 g, the exceeding of which have an effect on disorders of sugar, blood cholesterol and fat metabolism; and
inform the person (1) under examination if his or hers physical activity is preventing disorders in sugar, cholesterol and fat metabolism.

7. The transducer unit according to claim 6, characterized in that if the number of acceleration maxima performed has exceeded a given threshold level a whereby the physical activity has prevented disorders in sugar, cholesterol and fat metabolism.

8. The transducer unit according to the claim 7, characterized in that the maximum acceleration values are divided into the accelerations classes 0.3-0.5 g; 0.5-0.7 g; 0.7-0.9 g; 0.9-1.1 g and >1.1 g.

9. The transducer unit according to claim 8, characterized in that each registered maximum value of acceleration adds the occurrence by one (N=1) at the acceleration class it belongs to.

10. The transducer unit according to claim 9, characterized in that when the daily number of acceleration (N) maxima exceeds a given threshold level 6520 in the acceleration class 0.3-0.7 the physical activity performed has reduced blood cholesterol, triglycerides, and visceral fat.

11. The transducer unit according to claim 10, characterized in that it is configured to produce graphic and/or audiovisual presentation about the g-values, number of g-values, walking speed and energy expenditure on-line, as integrals of day, week or other time period and also information if the performed physical activity reach the given threshold level the exceeding of which reduces blood cholesterol, triglycerides and visceral fat.

12. The transducer unit according to claim 5 characterized in that it is an element of a cellular phone, by which physical activity of the person (1) is followed and steered to reduce blood cholesterol, triglycerides and visceral fat.

13. A computer program product, characterized in that it comprises computer code means saved on a computer readable media, which computer code means are configured to execute method steps of claims 1-5 by executing said computer program in a processor unit.
